(19) United States
(12) Patent Application Publication
(10) Pub. No.: US 2026/0317402 A1
(43) Pub. Date: Oct. 15, 2026

(54) Immunoglobulin preparations for treatment of systemic sclerosis

(71) Applicant: CSL Behring AG, Bern (CH)

(72) Inventors: Maria Gasior, King of Prussia, PA (US); Amgad Shebl, Marburg (DE); John-Philip Lawo, Marburg (DE); Adrian Panaite, Bern (CH); Orell Mielke, Marburg (DE)

(73) Assignee: CSL Behring AG, Bern (CH)

(21) Appl. No.: 19/482,564

(22) PCT Filed: May 8, 2024
(86) PCT No.: PCT/EP2024/062666

§ 371 Date: Nov. 7, 2025

Related U.S. Application Data
(60) Provisional application No. 63/500,679, filed on May 8, 2023.

(51) Int. Cl. C07K 16/06; A61K 9/00; A61K 39/00; A61P 37/06; C07K 16/18

(52) U.S. Cl. CPC C07K 16/06; A61K 9/0019; A61P 37/06; C07K 16/18; A61K 2039/505; A61K 2039/545; C07K 2317/21

(57) ABSTRACT

This disclosure pertains to the use of immunoglobulin preparations, such as liquid polyclonal immunoglobulin preparations for treatment of systemic sclerosis (SSc), such as diffuse cutaneous systemic sclerosis (dcSSc) and limited cutaneous systemic sclerosis (lcSSc). In some embodiments, the preparations are administered subcutaneously (SCIG preparations). In some embodiments, the preparations have a high concentration of immunoglobulin, such as 15-30% or 16-22% w/v.

### FIELD

This disclosure pertains to the use of immunoglobulin preparations, such as liquid polyclonal immunoglobulin preparations for treatment of systemic sclerosis (SSc), such as diffuse cutaneous systemic sclerosis (dcSSc) and limited cutaneous systemic sclerosis (lcSSc). In some embodiments, the preparations are administered subcutaneously (SCIG preparations). In some embodiments, the preparations have a high concentration of immunoglobulin, such as 15-30% or 16-22% w/v.

### BACKGROUND

Systemic sclerosis (SSc), also known as scleroderma, is an autoimmune connective-tissue disease. SSc is characterized by vascular damage (vasculopathy) followed by defective neovascularization and impaired vessel remodeling, and fibrosis of the skin and various organ systems (e.g., lung, renal, cardiac, gastrointestinal, and musculoskeletal). The disease has poor prognosis, and treatment options have been limited. Skin fibrosis is a major diagnostic feature of SSc, and the extent of skin involvement is used to stratify patients into two main subsets—limited cutaneous systemic sclerosis (lcSSc) and diffuse cutaneous systemic sclerosis (dcSSc). In contrast to scSSc and dcSSc, SSc without scleroderma is an uncommon presentation of SSc with classic internal organ involvement, but no skin thickness—and occurs in less than 5% of individuals with SSc. In general, patients with lcSSc exhibit skin fibrosis distal to the elbows and knees, whereas in dcSSc involvement occurs proximally to the elbows and knees and may involve a majority of the extremity. Patients with dcSSc are at an increased risk of complications such as scleroderma renal crisis and interstitial lung disease (ILD), which can be life-threatening, although some patients with lcSSc can also develop these complications. SSc-ILD is the most common direct cause of death in SSc, surpassing renal crisis and SSc-associated pulmonary arterial hypertension (SSc-PAH).

Pathogenesis of SSc is mediated by immune and inflammatory cells. Vascular injury drives infiltration of mast cells, T lymphocytes, and macrophages into lesional tissues early on in the disease. This results in an unresolving pro-inflammatory and pro-fibrotic response mediated by myofibroblast differentiation and the production of cytokines such as interleukin-6 (IL-6) and transforming growth factor (TGF-β).

Therapeutic strategies for SSc can include immunosuppressive therapy, vascular modulation, and/or symptomatic management. However, because the skin in SSc patients thickens and hardens over time (i.e., skin fibrosis), it is unclear if treatments can be applied subcutaneously; thus, there is a need to determine whether subcutaneous treatments can be given safely and whether such treatments have adequate bioavailability in SSc patients.

### SUMMARY

Among the methods disclosed herein, is a method of treating systemic sclerosis (SSc) in a human subject, comprising subcutaneous administration of a liquid human polyclonal immunoglobulin preparation comprising 16-22% w/v immunoglobulin, wherein the preparation is administered once, twice, or three times per week in order to achieve a weekly dose of 0.1 g/kg to 1 g/kg (embodiment 1). Another method encompassed by the disclosure is a method of treating systemic sclerosis (SSc) in a human subject, comprising subcutaneous administration of a liquid human polyclonal immunoglobulin preparation comprising 16-22% w/v immunoglobulin, wherein the preparation is administered twice per week in order to achieve a weekly dose of 0.1 g/kg to 1 g/kg (embodiment 2).

Embodiment 3 comprises the method of embodiment 1 or 2, wherein the liquid human polyclonal immunoglobulin preparation may comprise 20-22% immunoglobulin. Embodiment 4 comprises the method of any one of embodiments 1-3, wherein the immunoglobulin is derived from pooled plasma of more than 1000 human donors. Embodiment 5 comprises the method of any one of embodiments 1-4, wherein the preparation comprises a stabilizer comprising an amino acid such as proline, glycine, or arginine. Embodiment 6 comprises embodiment 5, wherein the stabilizer comprises proline. The proline of embodiment 6 may optionally be at a concentration of 100-500 mM, such as 200-300 mM, such as 250 mM. Embodiment 7 comprises embodiment 5 or 6, wherein the stabilizer further comprises a surfactant, such as polysorbate 80 or polysorbate 20, such as at 5-50 mg/L, such as 8-30 mg/L. Embodiment 8 comprises the method of any one of embodiments 1-7, wherein the preparation has a pH of 4.6 to 5.2. Embodiment 9 comprises the method of any one of embodiments 1-8, wherein the preparation comprises no carbohydrate stabilizers, comprises no more than 50 μg/mL IgA, and/or comprises no preservatives.

In any one of embodiments 1-9, the SSc may be diffuse cutaneous systemic sclerosis (dcSSc) or limited cutaneous SSc (lcSSc) (embodiment 10). In embodiment 11, the method of any one of embodiments 1-9 is conducted, wherein the SSc is dcSSc. Embodiment 12 comprises the method of embodiment 10 or 11, wherein the subject does not have dcSSc at the subcutaneous infusion site. Embodiment 13 comprises the method of any one of embodiments 1-12, wherein the method further comprises administration of at least one additional therapeutic agent. Embodiment 14 comprises the method of embodiment 13, wherein the at least one additional therapeutic agent does not comprise an antibody therapeutic agent. Embodiment 14 comprises the method of embodiment 13, wherein the at least one additional therapeutic agent does not comprise an anti-CD20 antibody, and/or does not comprise an anti-IL6 and/or does not comprise an anti-IL6 receptor antibody. Embodiment 16 comprises the method of any one of embodiments 1-15, wherein the subject has not previously received treatment with an anti-CD20 antibody and/or wherein the subject has not previously received treatment with an anti-IL6 antibody and/or wherein the subject has not previously received treatment with an anti-IL6 receptor antibody. Thus, in some embodiments herein, the method of any one of embodiments 1-14 is conducted, wherein the subject is not treated with an anti-CD20 antibody and/or has not previously been treated with an anti-CD20 antibody. In some embodiments herein, the method of any one of embodiments 1-14 is conducted, wherein the subject is not treated with an anti-IL6 or anti-IL6 receptor antibody and/or has not previously been treated with an anti-IL6 or anti-IL6 receptor antibody. Embodiment 17 comprises the method of any one of embodiments 1-16, wherein the subject has not been diagnosed with a rheumatic autoimmune disease other than SSc.

Embodiment 18 comprises the method of any one of embodiments 1-17, wherein the preparation is administered twice per week in order to achieve a weekly dose of 0.5 g/kg. Embodiment 19 comprises the method of any one of embodiments 1-18, wherein the preparation is administered for a period of at least one month, at least 3 months, at least 4 months, at least 6 months, or at least one year. Embodiment 20 comprises the method of any one of embodiments 1-19, wherein the subject, prior to the start of treatment, does not have a history of dermatitis, eczema or psoriasis. Embodiment 21 comprises the method of any one of embodiments 1-20, wherein the subject has a modified Rodnan skin score of 15-45 prior to treatment with the preparation. Embodiment 22 comprises the method of any one of embodiments 1-21, wherein the subject experiences no more than a mild or moderate infusion site skin reaction following administration with the preparation. Embodiment 23 comprises the method of any one of embodiments 1-22, wherein the subject does not experience an infusion site reaction such as infusion site pain or infusion site swelling within the first month of treatment, or within the first three months of treatment. Embodiment 24 comprises the method of any one of embodiments 1-23, wherein the preparation is subcutaneously administered using an infusion pump device. Embodiment 25 comprises the method of any one of embodiments 1-24, wherein a volume of 10 mL to 100 mL is subcutaneously administered in each dose.

Embodiment 26 comprises the method of any one of embodiments 1-25, wherein the preparation is administered for a period of at least one month, and wherein the subject, after one month, after 3 months, or after 4 months of treatment:
Has a trough serum IgG concentration of at least 18 g/L, at least 19 g/L, or at least 20 g/L;
Has an mRSS value that is reduced by at least 3, at least 4, or at least 5, or that is reduced by at least 15%, at least 20%, or at least 25% compared to baseline;
Has a SHAQ VAS that is decreased by at least 5%, at least 10%, or at least 15% compared to baseline;
Has a PGA that is decreased by at least 5%, at least 10%, or at least 15% compared to baseline;
Has an MDGA that is decreased by at least 5%, at least 10%, or at least 15% compared to baseline;
Has reduced pain, swelling and/or friction in at least one joint compared to baseline; and/or
Does not have an increase in UCLA SCTC GI 2.0 score compared to baseline.

Furthermore, in some cases, in the methods of any one of embodiments 1-26, wherein the subject, after one month, after 3 months, or after 4 months of treatment:
Has reduced swelling or pain in or more joints;
Does not have an increase in the number of swollen joints, or has a decrease in the number of swollen joints (e.g., a decrease by one joint or at least one joint);
Does not have an increase in the number of tender joints, or has a decrease in the number of tender joints (e.g., a decrease by one joint or at least one joint);
Does not have a decrease by any more than 5% or by any more than 10% in the manual muscle test (MMT) total score, (i.e., the MMT total score increases, remains the same, or decreases by no more than 5% or by no more than 10%); and/or
Does not show a decrease in forced vital capacity (FVC), in percentage, of any more than 5%, or any more than 2%, or any more than 1% (i.e., FVC % increases, remains the same, or decreases by no more than 5%, no more than 2%, or no more than 1%).

The present disclosure further comprises embodiment 27, comprising a liquid human polyclonal immunoglobulin preparation according to any one of embodiments 1-9, for use in a method according to any one of embodiments 1, 2, or 10-26. The disclosure herein also encompasses embodiment 28, comprising use of a liquid human polyclonal immunoglobulin preparation according to any one of embodiments 1-9 in the preparation of a medicament for treating SSc according to any one of embodiments 1, 2, or 10-26.

It is to be understood that both the foregoing general description and the following more detailed description are exemplary and explanatory only and are not restrictive of the claims. References cited herein are incorporated by reference in their entireties.

### BRIEF DESCRIPTION OF THE DRAWINGS

FIG. 1 shows a schematic of a clinical trial in dcSSc subjects, described in the Examples herein. *Several PK samples to measure IgG trough concentrations were collected to assess the steady state of IgPro20 or IgPro10 and carry-over effects on treatment period 2 from treatment period 1. In addition, PK samples were frequently collected over the last dose period to fully characterise the PK of IgPro20 and IgPro10. dcSSc, diffuse cutaneous systemic sclerosis; EOT, end of treatment; IgG, immunoglobulin G; IgPro10, 10% intravenous human immunoglobulin, Privigen®, CSL Behring; IgPro20, 20% subcutaneous human immunoglobulin, Hizentra®, CSL Behring; PK, pharmacokinetics.

FIG. 2A shows the mean (SD) serum IgG concentration-time profiles of patients with dcSSc following the first infusion of the last cycle of IgPro20 (0.5 g/kg/week) or IgPro10 (2 g/kg/4 weeks split over 2-5 days). More specifically, it shows serum IgG concentration in g/L on a linear scale after treatment with IgPro10 (intravenous 10% immunoglobulin preparation; solid line) or IgPro20 (subcutaneous 20% immunoglobulin preparation; dashed line), at days after the start of infusion, as noted on the x-axis. As not all subjects provided PK samples at all timepoints, numbers above the x-axis reflect subjects with non-missing values; upper raw for IgPro10 and lower raw for IgPro20. Bars represent standard deviation; IgG, immunoglobulin G; IgPro10, 10% intravenous human immunoglobulin, Privigen®, CSL Behring; IgPro20, 20% subcutaneous human immunoglobulin, Hizentra®, CSL Behring; SD, standard deviation.

FIG. 2B shows the mean (SD) serum IgG concentration-time profiles of patients with dcSSc following the first infusion of the last cycle of IgPro20 (0.5 g/kg/week) or IgPro10 (2 g/kg/4 weeks split over 2-5 days). More specifically, it shows serum IgG concentration in g/L on a log-linear scale after treatment with IgPro10 (intravenous 10% immunoglobulin preparation; solid line) or IgPro20 (subcutaneous 20% immunoglobulin preparation; dashed line), at days after the start of infusion, as noted on the x-axis. Bars represent standard deviation. As not all subjects provided PK samples at all timepoints, numbers above the x-axis as shown in FIG. 2A reflect subjects with non-missing values.

### DESCRIPTION OF EXEMPLARY EMBODIMENTS

### Definitions

It is to be understood that this disclosure is not limited to the particular methodology, protocols, cell lines, animal species or genera, and reagents described, as such may vary. It is also to be understood that the terminology used herein is for the purpose of describing particular embodiments only, and is not intended to limit the scope of the present disclosure which will be limited only by the appended claims.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

As used herein, the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this disclosure belongs unless clearly indicated otherwise.

In this disclosure, “comprises,” “comprising,” “containing,” “having,” “includes,” “including,” and linguistic variants thereof have the meaning ascribed to them in U.S. Patent law, permitting the presence of additional components beyond those explicitly recited.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

As used herein, the terms “treat,” “treating,” “treatment,” and the like include reducing, managing, or ameliorating a disorder such as SSc, and/or at least one sign or symptom associated therewith, or slowing or halting the progression of the disorder or one or more of its signs or symptoms. It will be appreciated that, although not precluded, treating a disorder such as SSc does not require that the disorder or symptoms associated therewith be completely eliminated. It will be further appreciated that treatment is often initiated after onset of symptoms such as skin fibrosis, in particular after diagnosis of the disorder.

A “therapeutically effective amount” refers to an amount of a drug sufficient to provide a treatment effect in a human subject, such as to alleviate at least one symptom, reduce or slow the progression, or prevent the onset of at least one symptom of a disorder such as SSc.

A “subject” or “patient” or “individual” to be treated herein refers to a human, unless specifically indicated otherwise.

“Systemic sclerosis” (SSc) is a progressive connective tissue disorder that is generally characterized by vascular damage, immune dysregulation or autoimmunity, and fibrosis. The vascular damage symptoms include Raynaud's phenomenon, digital ulcers, hypertensive renal failure, cardiomyopathy, and pulmonary hypertension. The fibrosis generally manifests as skin thickening, gastrointestinal dysmotility, myocardial fibrosis, or pulmonary fibrosis. Often patients initially present with Raynaud's phenomenon followed by non-Raynaud's symptoms such as skin thickening within one to several years. Patients may be classified into subtypes of SSc based on the distribution of skin thickening. Subtypes include diffuse cutaneous systemic sclerosis, limited cutaneous systemic sclerosis, and in certain rare cases systemic sclerosis without skin involvement.

“Diffuse cutaneous systemic sclerosis” (dcSSc) refers to SSc patients that have skin thickening that includes areas proximal to elbows and knees, and thus frequently over an entire extremity. It is generally considered a more severe form of SSc and accounts for about 20-40% of SSc cases. It may also be characterized by early internal organ involvement and a high mortality rate.

“Limited cutaneous systemic sclerosis” (lcSSc) refers to SSc patients that have skin thickening limited to areas distal to the elbows and knees, face, and neck. It accounts for most of the remaining cases of SSc.

“Raynaud's phenomenon” refers to a condition of localized reduced blood flow to end arterioles, such as in the fingers or toes. As a result of the reduced blood flow, the effected body parts such as fingers or toes may turn pale or white and then in some cases blue, with in some cases numbness, cold, or pain. Blood flow may return after several minutes or hours, causing redness and burning in the effected body parts.

The terms “polyclonal immunoglobulin preparation” and “immunoglobulin preparation” as used herein refer to a composition comprising polyclonal antibodies. Such preparations may, in some embodiments, be obtained from plasma from multiple donors, i.e., “pooled plasma.” The donors are human and the preparations are for use in humans unless expressly stated otherwise herein. The immunoglobulin in these preparations is mostly in the IgG form, called either “IgG” or “immunoglobulin G” herein. Hence, “immunoglobulin preparations” herein may also interchangeably be referred to as “IgG preparations” or immunoglobulin G preparations” or “Ig preparations” in some cases. In some embodiments, low levels of IgM and/or IgA are also present. The term “immunoglobulin” standing alone refers to an antibody molecule.

The term “subcutaneous” when modifying an immunoglobulin preparation herein, i.e. “subcutaneous immunoglobulin preparation” or “subcutaneous IgG preparation” and like terms, means that the preparation is administered to a subject subcutaneously. Such preparations are also known by the abbreviation “SCIG.” In some embodiments, such subcutaneous immunoglobulin preparations have a higher polyclonal IgG concentration than the alternative “intravenous” immunoglobulin preparations, abbreviated IVIG, which are preparations that are administered intravenously. Examples of subcutaneous IgG preparations and intravenous IgG preparations herein include the subcutaneous IgG preparation IgPro20 and the intravenous IgG preparation IgPro10, referred to in the Examples below, among others.

As used herein, “relative bioavailability,” when referring to a “subcutaneous immunoglobulin preparation” means the relative amount of the immunoglobulin (provided as a fraction or percentage) that reaches the systemic circulation compared to administration of an intravenous immunoglobulin preparation, for which bioavailability is set at 100% (as it is directly administered to the bloodstream).

The term “baseline,” when referring to a score or index value, such as mRSS, HAQ DI, MDGA, PGA, or the like herein means a time before the start of treatment with a subcutaneous immunoglobulin preparation according to a method herein. Hence, if a score changes after a certain time of treatment compared to baseline, the baseline value of a particular index refers to the value of that index taken prior to the start of treatment.

A “stabilizer” as used herein refers to a molecule, such as an amino acid, a carbohydrate, a surfactant, a sugar alcohol, or a protein such as albumin, or the like, that may be added to a polyclonal immunoglobulin preparation in order to improve its shelf-life during storage, for example, by reducing aggregation, reducing oxidation, reducing discoloration, or the like.

A “surfactant” refers to a molecule that has an amphiphilic structure, such that it is hydrophilic on one end, such as through a polar or charged chemical group, and is hydrophobic at the other end, such as through carbohydrate groups. Examples include polysorbates, poloxamers, and various fatty acids and lipids.

A “preservative” is a substance that prevents the growth of bacteria or other microbes in a solution.

Additional definitions may be found in the sections that follow.

### A. Immunoglobulin Preparations

Immunoglobulin preparations herein include, for example, preparations of primarily immunoglobulin G that are administered intravenously or subcutaneously (abbreviated IVIG and SCIG, respectively). In some cases, the immunoglobulin preparation is in liquid form, i.e., it has not been lyophilized previously or has been reconstituted in liquid form after earlier lyophilization. In some cases, the preparation has not been subject to lyophilization previously. In some embodiments, the preparation is in “ready-to-use” format, meaning it can be directly infused or injected or otherwise administered to a subject without dilution, reconstitution, or the like.

In some cases, the immunoglobulin preparation is a relatively high concentration IgG preparation that is administered subcutaneously. In some embodiments, the preparation comprises at least 15% w/v, at least 20% w/v, at least 25% w/v, or at least 30% w/v immunoglobulin. In some cases, the concentration of immunoglobulin (i.e., IgG and other immunoglobulin forms such as IgA and IgM) in the preparation is 15-30% w/v. In some cases, the preparation comprises 16-25% w/v immunoglobulin. In some cases, the preparation comprises 16-22% w/v immunoglobulin. In some cases, the preparation comprises 18-22% w/v immunoglobulin. In some cases, the preparation comprises 20-22% w/v immunoglobulin. In some such cases, the preparation is a ready-to-use liquid preparation that is administered subcutaneously to the subject.

In some cases, the preparation is administered subcutaneously once, twice, or three times per week. In some cases, the weekly dose of the preparation is from 0.1 g/kg to 1 g/kg. In some cases, the weekly dose of the preparation is from 0.1 g/kg to 0.6 g/kg. In some cases, the weekly dose of the preparation is from 0.1 g/kg to 0.5 g/kg. In some cases, the weekly dose of the preparation is from 0.2 g/kg to 0.5 g/kg. In some cases, the weekly dose of the preparation is from 0.1 g/kg to 0.4 g/kg. In some cases, the weekly dose of the preparation is from 0.4 g/kg to 0.6 g/kg. In some cases, the weekly dose is 0.1 g/kg, 0.15 g/kg, 0.2 g/kg, 0.25 g/kg, 0.3 g/kg, 0.4 g/kg, 0.5 g/kg, 0.6 g/kg, 0.75 g/kg, or 1 g/kg. In some cases, the weekly dose of the preparation is 0.5 g/kg. In some cases, where a subject weighs more than 100 kg, a fixed weekly dose of 50 g of the preparation is administered. In some cases, according to the dosage regimes above, subcutaneous doses are administered twice per week, such as on day 1 and either day 3 or day 4 of each week.

In some embodiments, the immunoglobulin in the preparation is derived from pooled plasma of hundreds or thousands of individual human donors. For example, in some cases, the preparation is derived from pooled plasma of more than 1000 human donors.

Immunoglobulin preparations herein may be stabilized in various ways. For example, in some embodiments, the preparation comprises a stabilizer comprising an amino acid, such as proline, glycine, or arginine. In some cases, the amino acid is glycine. In other cases, the amino acid is proline. In some cases, the stabilizer consists essentially of an amino acid. In some cases, the stabilizer comprises or consists essentially of proline, optionally at a concentration of 100-500 mM, such as 200-300 mM, such as 250 mM. In some cases, the stabilizer further comprises a surfactant, such as polysorbate 80 or polysorbate 20. In some embodiments a polysorbate surfactant is provided at a concentration of 100 mg/L or less, or 70 mg/L or less, such as at 5-50 mg/L, such as 8-30 mg/L. In some embodiments, the stabilizer comprises both an amino acid and a surfactant. In some embodiments, the stabilizer consists essentially of both an amino acid and a surfactant. In some cases, the stabilizer comprises or consists essentially of proline and a surfactant. In some such cases, the concentration of proline is 100-500 mM, such as 200-300 mM, such as 250 mM and the surfactant is polysorbate 80 or polysorbate 20 at a concentration of 5-50 mg/L, such as 8-30 mg/L. In some embodiments, the preparation has a pH of 4.6 to 5.2, such as 4.8 to 5.2. In some cases, the preparation comprises no carbohydrate stabilizers. In some cases, the preparation comprises no carbohydrate, sugar alcohol, or protein stabilizers (such as albumin). In some cases, such as where the stabilizer consists essentially of an amino acid or consists essentially of an amino acid and surfactant, the preparation still may contain trace amounts of salts such as sodium or potassium, peptides, or other components that are found in trace amounts either in the immunoglobulin or in the stabilizers. In some cases, the preparation does not comprise any preservatives. In some cases, a liquid polyclonal immunoglobulin preparation has never been lyophilized.

In some cases, the immunoglobulin of the preparation is at least 98% IgG. In some cases, the immunoglobulin subclass distribution is 60-75% IgG1, 20-35% IgG2, 2-5% IgG3, and 1-3% IgG4. In some cases, the subclass distribution is 62-74% IgG1, 22-34% IgG2, 2-5% IgG3, and 1-3% IgG4. In some cases, the preparation comprises no more than 100 μg/mL IgA, or no more than 50 μg/mL IgA. In some cases, the content of IgA is no more than 100 μg per 100 mg immunoglobulin, or no more than 50 μg per 100 mg immunoglobulin, or no more than 25 μg per 100 mg immunoglobulin. In some cases, the preparation comprises no carbohydrate stabilizers, comprises no more than 50 μg/mL IgA, and/or comprises no preservatives. In some cases, the preparation comprises no carbohydrate stabilizers, comprises no more than 50 μg/mL IgA, and comprises no preservatives. In some cases, the preparation comprises at least 98% IgG, proline at 200-300 mM (or 250 mM), polysorbate 80 at 8-30 mg/L, trace sodium, and no more than 50 μg/mL IgA, and no preservatives.

Examples of immunoglobulin preparations suitable for the present subcutaneous administration methods include, for example the subcutaneous immunoglobulin preparations Hizentra® (immune globulin subcutaneous), which is a 20% liquid ready to use immunoglobulin preparation to be administered subcutaneously, which comprises at least 98% IgG, proline at 200-300 mM (or 250 mM), polysorbate 80 at 8-30 mg/L, trace sodium, and no more than 50 μg/mL IgA, and has a pH of 4.6 to 5.2. (Hizentra® FDA Package Insert, 2021.) Other example subcutaneous immunoglobulin preparations include Cuvitru® and Vivaglobin®, for example. For example, Vivaglobin® comprises 16% w/v immunoglobulin, and comprises glycine as a stabilizer, and further comprises sodium chloride, with a pH of 6.4 to 7.2. (Vivaglobin® FDA package insert.) Cuvitru®, for example, comprises 20% immunoglobulin, and the immunoglobulin of the preparation is at least 98% IgG. It comprises 250 mM glycine as a stabilizer, and a pH of 4.6 to 5.2 (e.g., 4.6 to 5.1). It comprises an IgA content of no more than 100 μg/mL. (Cuvitru® 2021 FDA package insert.)

Intravenously administered immunoglobulin preparations, by contrast, include, for example, Privigen® and Sandoglobulin®/Carimune®, for example, as well as Gamunex-C®, Gammagard®, and Octagam®. Privigen®, for example, comprises 10% w/v immunoglobulin along with a purity of at least 98% IgG monomers, and further containing 250 mM L-proline (210-290 mM range), trace sodium, and no carbohydrate stabilizers and no preservative, with a pH of 4.8 (4.6-5.0 range) and less than or equal to 25 μg/mL IgA. (Privigen® FDA package insert 2017, version 56.)

In some embodiments, the preparation is provided in a single-dose container, such as a prefilled syringe, e.g., at 5 mL, 10 mL, 20 mL, and 50 mL; or in a single-dose tamper-resistant vial, e.g., at 5 mL, 10 mL, 20 mL, and 50 mL. In some cases, the preparation is administered using an infusion pump device. In some cases, the total volume of fluid administered to the subject in each dose is between 10 mL and 100 mL, such as between 50 mL and 100 mL, or between 25 mL and 75 mL.

### B. Treatment of Systemic Sclerosis and Administration of Subcutaneous Immunoglobulin Preparations

SSc comprises two main subsets—limited cutaneous systemic sclerosis (lcSSc) and diffuse cutaneous systemic sclerosis (dcSSc). Both conditions are characterized by skin fibrosis—a thickening and hardening of the skin. Patients with lcSSc may exhibit skin fibrosis which is distal to the elbows and knees, such as on the hands, face, feet, and/or forearms, and may experience symptoms such as abnormal skin pigmentation and texture, and Raynaud's phenomenon. Patients with dcSSc exhibit skin fibrosis proximal to the elbows and knees, and thus that may extend over most of or an entire extremity. Patients with dcSSC also may experience symptoms such as Raynaud's phenomenon, gastroesophageal reflux, difficulty eating (dysphagia), and in some cases complications involving the lung and/or kidneys, including pulmonary fibrosis and renal insufficiency.

The mechanism of action of polyclonal immunoglobulin preparations such as those described here is complex and not fully understood, but may involve an increase in the overall concentration of IgG antibodies, which may in part block interactions between disease-related autoantibodies and Fcgamma receptors or other targets. In general, autoantibodies that interact with Fcgamma receptors are often found in patients with autoimmune disease, including SSc patients. Fcgamma receptors are surface glycoproteins that modulate the coordination between the activating and inhibitor signals of immune cells. One possible treatment strategy, without being bound by theory, that may be applied to SSc patients is to target Fcgamma receptors. Thus, targeting Fcgamma receptors may help to attenuate inflammation and autoimmunity in SSc.

Methods herein involve subcutaneous administration of a polyclonal immunoglobulin preparation, such as described in the section above, to a subject with SSc, such as dcSSC or lcSSc. In some cases, the SSc is diffuse cutaneous systemic sclerosis (dcSSc) or limited cutaneous SSc (lcSSc). In some cases, the SSc is dcSSc.

Because skin fibrosis causes the skin to thicken and harden over time, it was not certain whether polyclonal immunoglobulin preparations such as those herein could be safely administered subcutaneously to SSc patients without causing either reactions at the site of subcutaneous infusion or low bioavailability due to the difficulty in penetrating diseased skin. For example, when immunoglobulin preparations are administered subcutaneously to treat other conditions, adverse effects may include local infusion site reactions, rash, local heat, local pain, and itching, among others. In some embodiments, 1, 2, 3, 4, 5, 6, 7, or 8 infusion sites are used simultaneously for subcutaneous administration of the treatment. In some embodiments, these infusion sites are situated such that each site is at least two inches away from another site. In some cases, infusion sites are located on the abdomen, thighs and/or lateral hip of the subject. In some cases, the subject with SSc does not have skin thickening at the subcutaneous infusion site or sites. For example, the subject may show evidence of skin thickening at other areas of the body. However, where multiple sites are needed, this may not necessarily be possible. Thus, in some cases, the site chosen for subcutaneous administration does not display evidence of skin thickening, although other areas of the body may show evidence of skin thickening. Thus, in other cases, the subject does have skin thickening at least one subcutaneous infusion site. For example, in some cases, the subject has no more than mild or moderate skin thickening at the infusion site or sites, such as an mRSS score of 2 or lower. In some cases, prior to the start of treatment, the subject does not have a history of dermatitis, eczema or psoriasis, or does not have a history of dermatitis, eczema or psoriasis at the subcutaneous infusion site or sites.

In cases where a preparation is administered subcutaneously, the preparation may be self-administered by the subject, for example, using an infusion pump or similar device. For a dosage of 0.1-1 g/kg, for example, each week, the volume of the subcutaneous infusion may be on the order of 10 mL to 100 mL per week, for example, such as 10-50 mL or 10-20 mL, or 20-50 mL. In some cases, the preparation is administered for a period of at least two weeks, at least one month, at least 3 months, at least 6 months, at least 9 months, or at least one year.

In some cases, the method further comprises administration of at least one additional therapeutic agent. In some cases, the at least one additional therapeutic agent does not comprise an antibody therapeutic agent. In some such cases, the at least one additional therapeutic agent does not comprise an anti-CD20 antibody, and/or does not comprise an anti-IL6 or anti-IL6 receptor antibody. In some cases, the subject has not previously received treatment with an anti-CD20 antibody and/or the subject has not previously received treatment with an anti-IL6 antibody and/or the subject has not previously received treatment with an anti-IL6 receptor antibody. In other cases, an antibody therapeutic has previously been administered to the subject. Examples of an antibody therapeutic agent include nintedanib, tocilizumab, clazakizumab, and rituximab. Thus, in some cases, the subject does not receive therapeutic treatment with any of nintedanib, tocilizumab, clazakizumab, and rituximab along with the immunoglobulin preparation. In other cases, the subject has previously been treated with one or more of nintedanib, tocilizumab, and rituximab or with an anti-CD20 or anti-IL6 or anti-IL6 receptor antibody.

In some embodiments, the subject is treated with at least one additional therapeutic agent comprising an immunosuppressant. In some embodiments, the subject is treated with at least one additional therapeutic agent comprising a small molecule immunosuppressant. In some embodiments, the subject is also treated with cyclophosphamide, methotrexate, mycophenolate mofetil (MMF), and/or autologous stem cell transplant (aSCT). For example, agents such as cyclophosphamide, methotrexate, or mycophenolate mofetil can be administered as an additional therapeutic agent for treatment of fibrosis symptoms such as skin sclerosis and SSc-ILD. In some embodiments, the at least one additional therapeutic agent comprises an angiotensin-converting enzyme inhibitor and/or an endothelin receptor antagonist. In some embodiments, the at least one additional therapeutic agent comprises ambrisentan. In some embodiments, the at least one additional therapeutic agent may treat symptoms related to interstitial lung disease in SSc (SSc-ILD). In some embodiments, the at least one additional therapeutic agent comprises a phosphodiesterase type 5 inhibitor. In some embodiments, the at least one additional therapeutic agent is tadalafil.

In some cases, the subject has not been diagnosed with a rheumatic autoimmune disease other than SSc. For example, in some cases, the subject has not been diagnosed with one or more of rheumatoid arthritis, lupus, mixed connective tissue disorder, polymyositis, dermatomyositis, Sjogren's syndrome, myositis, or primary biliary cholangitis. In some cases, the subject has not been diagnosed with one or more of rheumatoid arthritis, lupus, mixed connective tissue disorder, polymyositis, or dermatomyositis. In other cases, the subject has been diagnosed with at least one other rheumatic autoimmune disease. In some cases, the systemic sclerosis autoantibodies have been detected in the subject. In some cases, the subject, prior to the start of treatment, does not have a history of dermatitis, eczema or psoriasis at the site or sites of subcutaneous infusion.

Potential adverse events associated with subcutaneous administration of an immunoglobulin preparation include infusion site pain, swelling, or inflammation. In some cases, the subject herein experiences no more than a mild or moderate infusion site skin reaction following administration with the preparation. In some cases, the subject does not experience an infusion site reaction such as infusion site pain or infusion site swelling within the first month of treatment, or within the first three months of treatment.

C. Efficacy, Bioavailability and Pharmacokinetics of Treatment with Subcutaneous Immunoglobulin Preparations

As described in the Examples below, a clinical trial was performed comparing administration of an intravenous IgG preparation IgPro10 (comprising 10% w/v immunoglobulin) to that of a subcutaneous IgG preparation IgPro20 (comprising 20% w/v immunoglobulin) in 27 human subjects with SSc. As described in the Examples below, subjects were either treated intravenously with IgPro10 followed by subcutaneously with IgPro20, for a period of 17 weeks each, or were treated subcutaneously with IgPro20 followed by intravenously with IgPro10, for a period of 17 weeks each. On average, in the 27 subjects, the population relative bioavailability of subcutaneous IgG was 76% (with a standard deviation of about 8%; based on dose-normalized, baseline-corrected AUC₀₋ₜₐᵤ), compared to intravenous IgG (which is set at 100%). Specifically, when IgPro20 was administered first in the sequence, its relative bioavailability compared to IgPro10 was 83% on average, whereas when IgPro20 was administered second in the sequence, its relative bioavailability compared to IgPro10 was 70% on average. The term “AUC₀₋ₜₐᵤ” refers to the area under the curve (AUC) measured from the beginning to the end of the dosing period, i.e., during the entire dosing period. Accordingly, the relative bioavailability of the subcutaneous IgG preparation in some embodiments herein may be at least 65%, at least 70%, at least 75%, or at least 80% compared to an intravenous IgG preparation administered intravenously at the same overall dosage in the subject, which is set at 100%. In some embodiments, this relative bioavailability is calculated as (AUC₀₋ₜₐᵤ SC/dose of SC)/(AUC₀₋ₜₐᵤ of IV/dose of IV), where SC is the subcutaneous immunoglobulin preparation and IV is the comparative intravenous immunoglobulin preparation.

In the Examples below, the IgPro10 and IgPro20 comparative study also compared various parameters related to the pharmacokinetics and efficacy of the subcutaneous and intravenous preparations in the treated subjects.

For example, the serum IgG concentration was determined in subjects throughout the dosing period in order to calculate the mean trough serum IgG concentration, among other parameters, for all of the subjects in each group in the trial. It was found that subcutaneous administration of the immunoglobulin preparation led to a higher trough serum IgG concentration. Thus, in methods herein, for example, the subject may have a trough serum IgG concentration following subcutaneous administration of an immunoglobulin preparation of at least 18 g/L, at least 19 g/L, or at least 20 g/L. In some cases, the subject may have a trough serum IgG concentration following subcutaneous administration of an immunoglobulin preparation ranging from 18 to 25 g/L, from 19 to 25 g/L, from 20 to 25 g/L, from 20 to 24 g/L, or from 21 to 24 g/L. In some subjects the trough serum IgG concentration ranges from or 22 to 24 g/L or from 21 to 23 g/L or from 21 to 22 g/L. In contrast, the mean trough serum IgG concentration following intravenous administration of an immunoglobulin preparation for the subjects assayed in the trial was 17-18 g/L. Baseline serum IgG concentration for the subjects was 12-13 g/L.

Subjects receiving treatment according to methods herein may also show improvements in several efficacy measures, as shown, for example, in Tables 3 and 4 below. For example, in some cases, prior to administration, the subject's skin thickness is assessed and a modified Rodnan skin score (mRSS) calculated for the subject. (P. J. Clements et al, J. Rheumatol. 20(11): 1892-6 (1993); D. Khanna et al., JSRD 2(1): 11-18 (2017).) The modified Rodnan skin score (mRSS) is a physician-performed assessment measure often used to examine progression of cutaneous fibrosis (i.e., skin thickening) over time. The mRSS is a semiquantitative score. The mRSS evaluates skin thickness at 17 different cutaneous sites, with a score at each site ranging from 0 (for normal), 1 (mild skin thickness), 2 (moderate skin thickness), or 3 (for severe or thick), for a total score ranging from 0 to 51, in which a higher score indicates thicker skin. Since change in mRSS score is correlated with histological skin thickness, it is often used as a primary or secondary outcome in clinical trials. The mRSS not only may provide a means to monitor the impact of potential therapies on skin thickness, but also a way to evaluate if skin thickness may interfere with subcutaneous administration of treatments.

In some cases, the subject, prior to administration, has a modified Rodnan skin score (mRSS) of at least 15 prior to treatment with the preparation, and in some cases has an mRSS score of 15-45 prior to treatment with the preparation. In some cases, infusion sites are chosen from sites having an mRSS score of 2 or lower. In some cases, treatment with the preparation herein reduces the mRSS for the patient compared to the score prior to administration. In some subjects tested, the mRSS was reduced from in value by 3-8 points following at least 17 weeks of treatment with subcutaneous IgG preparations, where the starting mRSS was between 20 to 25. Thus, for example, in some embodiments, the subject's starting mRSS value is reduced by at least 3, at least 4, at least 5, at least 6, at least 8, or at least 10 after at least 1, 2, 3, or 4 months of treatment. In some embodiments, the starting mRSS value is reduced by 2-10, 2-8, 3-8, 2-5, 3-10, 4-10, 5-10, 4-8, or 5-8 after at least 1, 2, 3, or 4 months of treatment according to the methods herein. In some embodiments, the subject has a decrease of at least 15%, at least 20%, or at least 25% in mRSS after at least 1, 2, 3, or 4 months of treatment according to methods herein.

For example, as described in the Examples below, out of 27 subjects, 11 (40.7%) subjects showed a decrease in mRSS of at least 5 points or at least 25% in after 1 to 17 weeks of treatment, and 18 (66.7%) showed a decrease in mRSS of at least 5 points or at least 25% over weeks 1 to 32. Out of 27 subjects, 13 (48.1%) subjects showed a decrease in mRSS of at least 4 points or at least 20% in weeks 1 to 17, and 20 (74.1%) showed a decrease in mRSS of at least 4 points or at least 20% over weeks 1 to 32.

In the Examples below, patients also showed a decrease (improvement) in mean Scleroderma Heath Assessment Questionnaire (SHAQ) overall disease scores measured on a visual analog scale (SHAQ VAS) and HAQ disability index (HAQ DI) scores. SHAQ consists of both the HAQ-DI and the scleroderma-specific VAS items. The SHAQ VAS is a self-reported questionnaire focusing on quality of life for an SSc subject, and focusing on the following items: pain, intestinal disease, breathing problems, Raynaud's syndrome, finger ulcers, and overall disease. Each VAS item is rated separately, with a higher score indicating more severe disease. (See e.g., J. Pope, Arthritis Care & Research, 63(S11), pp. S98-S110 (2011).) In some examples, the SHAQ VAS after 17 weeks of treatment decreased by at least 5%, at least 10%, at least 15%, or at least 20%, when compared to baseline prior to treatment. For instance, the SHAQ VAS, which has a scale of from 0 to 3, where higher numbers indicate worse patient condition, in the trial had a mean value of 1.2 prior to treatment and of 1.0 to 1.1 after 17 or 32 weeks of treatment. Thus, in some cases, a patient's SHAQ VAS score after at least 1, 2, 3, or 4 months of treatment according to methods herein may decrease by at least 5%, at least 10%, at least 15%, or at least 20%. In some cases, a subject may also have an improvement (i.e. reduction) in the HAQ DI score. HAQ-DI is a self-reported questionnaire of 20 questions referring to 8 domains: dressing/grooming, arising, eating, walking, hygiene, reach, grip, and activities, where the total score indicates the level of self-assessed disability. Each question is graded on a scale of 0 (no difficulty), 1 (some difficulty), 2 (much difficulty), or 3 (unable to do). The total score is the sum of the domain scores, divided by the number of domains that have a score, based on this range of 0-3, with higher total scores indicating greater disfunction. (J. F. Fries et al., Arthritis Rheum., 23(2): 137-45 (1980); V. Steen & T. A. Medsger, Jr., Arthritis Rheum. 44: 2828-35 (2001); J. Pope, supra; C. Almeida et al., Autoimmun. Rev. 14: 1087-96 (2015).) In some cases, a patient's HAQ DI score after at least 1, 2, 3, or 4 months of treatment according to methods herein may also decrease by at least 5%, at least 10%, at least 15%, or at least 20%.

In some cases, a UCLA (University of California at Los Angeles) Scleroderma Clinical Trial Consortium Gastrointestinal Tract score is assessed for a subject (UCLA SCTC GIT 2.0 score). This is a self-reported score based on gastrointestinal tract symptoms for SSc patients. (See, e.g., D. Khanna et al., Arthritis Rheum., 61(9): 1257-63 (2009); D. Khanna et al., J. Rheumatol., 38(9): 1920-24 (2011); G. Abignano et al., J. Rheumatol. 48(9): 1422-26 (2021).) The survey consists of 34 questions on items as follows: reflux (1-8), distention/bloating (9-12), fecal soilage (13), diarrhea (14-15), social functioning (16-21), emotional well-being (22-30), constipation (31-34). The items are scored on a scale of 0, 1, 2, or 3, where 0 indicates better health and higher numbers indicate worse health. Scores from each subset except constipation are combined to form a total GIT score to capture overall burden of severity of SSc-associated GIT. In the tested subjects, the GIT scores did not increase on average over the course of the treatment. In some cases, the UCLA SCTC GIT 2.0 score does not increase by any more than 15% or does not increase by any more than 10%, or decreases after at least 1, 2, 3, or 4 months of treatment according to methods herein.

In some examples, following at least 1, 2, 3, or 4 months of treatment according to the methods herein, the subject has a decrease (improvement) on the Physician Global Assessment test (MDGA). MDGA is rated by the physician and MDGA values range from 0 to 10, with higher scores indicating worse patient condition. In some examples, the MDGA value is decreased after at least 1, 2, 3, or 4 months of treatment according to methods herein by at least 5%, at least 10%, at least 15%, or at least 20%, when compared to baseline before treatment with the immunoglobulin preparation.

In some examples, following the treatment of at least 1, 2, 3, or 4 months according to methods herein, the subject has a decrease (improvement) on the Patient General Assessment test (PGA), which is a patient self-reported test. The PGA also is scored on a scale of 0 (excellent) to 10 (extremely poor), with higher scores indicating worse disease. In some examples, the PGA value is decreased by at least 5%, at least 10%, at least 15%, or at least 20% over this time period, when compared to baseline.

In some cases, a subject treated according to methods herein, after at least 1, 2, 3, or 4 months of treatment, shows improvement in at least one of mRSS, SHAQ-DI and or VAS score, UCLA SCTX GIT 2.0 score, PGA, or MDGA, such as by at least 5%, at least 10%, at least 15%, or at least 20% compared to the score before treatment (baseline). In some cases, methods herein result in an improvement in one or more of mRSS Total Score, HAQ-DI and VAS scores, PGA, and MDGA, by at least 5%, at least 10%, at least 15%, or at least 20% compared to before treatment (baseline), after at least 4 weeks, or at least 8 weeks, or at least 12 weeks, or at least 16 weeks of treatment.

In some cases, swelling or pain in one or more joints is assessed in a subject. For example, swelling in joints such as the proximal interphalangeal joints of the fingers, metacarpophalangeal joints, wrists, elbows, shoulders, and/or knees may be assessed by a physician and assessed as either swollen or not swollen. These same joints may also be assessed for tenderness as either tender or not tender. In some cases, swelling or tenderness in or more joints is reduced after at least 1, 2, 3, or 4 months of treatment according to methods herein. For example, in some cases, the number of swollen joints does not increase after at least 1, 2, 3, or 4 months of treatment according to methods herein. In some cases the number of swollen joints decreases, e.g., decreases by one joint, after at least 1, 2, 3, or 4 months of treatment according to methods herein. For example, in some cases, the number of tender joints does not increase after at least 1, 2, 3, or 4 months of treatment according to methods herein. In some cases the number of tender joints decreases, e.g., decreases by one joint, after at least 1, 2, 3, or 4 months of treatment according to methods herein. In some patients, a qualified individual may also perform a manual muscle test (MMT), for example testing the neck flexors (axial) and the following bilateral, proximal muscles (deltoid middle, biceps brachii, gluteus maximus, gluteus medius, quadriceps, and the following distal muscles: wrist extensors and ankle dorsiflexors. These assessments in some cases may be used to derive an overall MMT total score. In some cases, the MMT total score does not increase after at least 1, 2, 3, or 4 months of treatment according to methods herein, and in some cases does not decrease by more than 5% or by more than 10% after at least 1, 2, 3, or 4 months of treatment according to methods herein (i.e., increases, remains the same, or decreases by no more than 5% or by no more than 10%).

In some cases, forced vital capacity (FVC) predicted, in percentage, is determined for a subject. The “FVC % predicted” measures the adjusted volume of air (adjusted as a percentage of the expected normal value based on the patient's age, height, gender, and ethnicity) that can be forcibly exhaled from the lungs after taking the deepest breath possible. In some such cases, the subject does not show a decrease in FVC % of any more than 5%, or any more than 2%, or any more than 1% after at least 1, 2, 3, or 4 months of treatment according to methods herein.

The following examples are put forth so as to provide those of ordinary skill in the art with a complete disclosure and description of how to make and use the subject disclosure, and are not intended to limit the scope of what is regarded as the disclosure. Efforts have been made to ensure accuracy with respect to the numbers used (e.g., amounts, temperature, concentrations, etc.) but some experimental errors and deviations should be allowed for. The entire disclosure of each document cited (including patents, patent applications, journal articles, abstracts, manuals, books, or other disclosures) is herein incorporated by reference in its entirety.

### EXAMPLES

### Example 1. Safety and Pharmacokinetics of IgPro20 (Subcutaneous Immunoglobulin) and IgPro10 (Intravenous Immunoglobulin) in Adults with Systemic Sclerosis

This example relates to evaluating local infusion safety, bioavailability, and pharmacokinetics (PK) of subcutaneous IgG (SCIG) (IgPro20) in adults with diffuse cutaneous SSc (dcSSc).

### A. Summary of the Study and Results

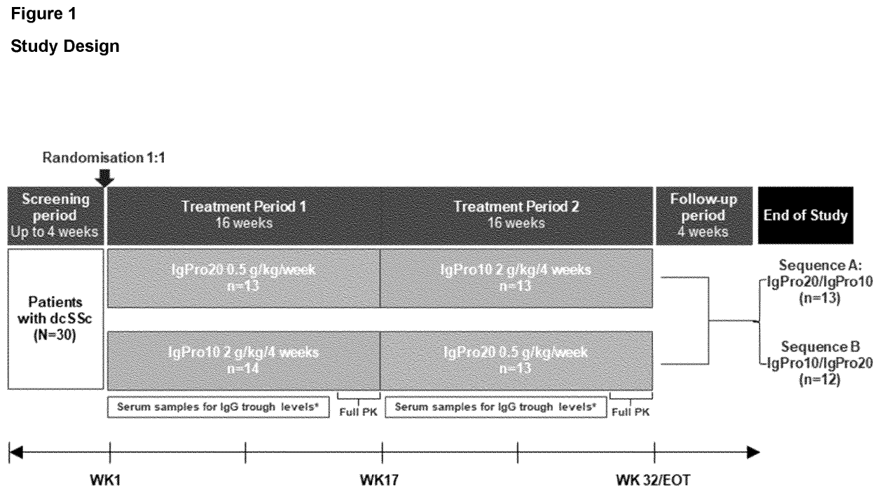

A randomized, open-label, crossover study was designed (NCT04138485). Adult subjects with (1) dcSSc diagnosis within 5 years from first non-Raynaud's phenomenon and (2) modified Rodnan Skin Score of 15-45 at screening were randomized 1:1 to receive one of two treatment sequences: (1) sequence A (IgPro20, 20% normal human subcutaneous immunoglobulin followed by IgPro10, 10% normal human intravenous immunoglobulin), or (2) sequence B (IgPro10 followed by IgPro20). A total of 27 subjects were randomized, with 13 subjects assigned to sequence A and 14 subjects assigned to sequence B. FIG. 1 provides the overall study design.

Each subject completed two treatment periods of 16 weeks each, with up to 40 weeks (including screening) of study duration for an individual subject. Doses received were 0.5 g/kg/week split over two sessions for IgPro20, and 2 g/kg/4 weeks split over 2-5 days for IgPro10. The primary endpoint was safety of IgPro20, described as treatment-emergent adverse events (TEAEs) and changes in clinical observations. Secondary endpoints included the proportion of subjects meeting cardiopulmonary or renal failure criteria in ACR CRISS, proportion of responders (with ACR CRISS of >0.6), and mean change in modified Rodnan skin score, for example, over a 48 week period.

Exclusion criteria for subjects also included, for example, diagnosis of primary rheumatic disease other than dcSSc, including rheumatoid arthritis, systemic lupus erythematosus, mixed connective tissue disorder, polymyositis, dermatomyositis. Subjects with fibromyalgia, secondary Sjogren's syndrome, and scleroderma-associated myopathy or myositis were not excluded. Further exclusion criteria included positive anti-centromere antibodies; evidence of severe chronic kidney disease or patients receiving dialysis; history of thrombotic episodes such as myocardial infarction or stroke, documented thrombophilic abnormalities; three or more of: presence of atrial fibrillation, coronary disease, diabetes mellitus, dyslipidemia, hypertension, or obesity; presence bacterial or viral infection; malignancies; known hypoalbuminemia; and known IgA deficiency.

The study results, described in more detail in the next sections, indicate that subcutaneous administration of IgPro20 has acceptable safety, bioavailability, and PK profiles in patients with dcSSc.

Of the 27 treated subjects, 25 subjects completed the study. 107 treatment emergent adverse events (TEAEs) occurred in 22 subjects (81.5%) over the 36-week study period, the majority of which were mild or moderate. The most common TEAEs (>10% of subjects) by preferred term (PT) were headache (12 events occurring in 6 subjects [22.2%]), COVID-19 (3 events occurring in 3 subjects [11.1%]), diarrhea (3 events occurring in 3 subjects [11.1%]), and vomiting (3 events occurring in 3 subjects [11.1%]). A total of 10 serious adverse events (SAEs) were reported in 6 subjects (viral infection, chronic gastritis, vomiting, dehydration, upper gastrointestinal haemorrhage, chest pain, myocardial infarction, myocardial ischemia, breast cancer, interstitial lung disease). Among these, one subject experienced 2 SAEs (myocardial ischemia & myocardial infarction) and was discontinued from study treatment. None of the SAEs were considered related to study treatment by the investigator, and no deaths were reported.

For IgPro20, 14 infusion site reactions (ISRs) occurred in 5 subjects (19.2%); all were mild or moderate in severity. The most common ISRs were infusion site pain and infusion site swelling (3 events in 2 subjects each, 7.7%). In total, 686 IgPro20 infusions were performed, resulting in an overall ISR rate per infusion of 0.02, i.e., 2 ISRs per 100 infusions. No ISRs were reported for IgPro10.

No clinically relevant trends in vital signs, body weight, clinical laboratory tests, electrocardiograms, or pulmonary function tests were observed. PK profiles and bioavailability in dcSSc subjects were similar to those observed in other approved indications, such as primary immunodeficiency. Population relative bioavailability of IgPro20, based on dose-normalized, baseline-corrected AUC₀₋ₜₐᵤ was 0.761 (90% CI: 0.7033, 0.8232), ie 76.1% compared to IgPro10 (intravenous IgG).

The overall safety profiles of IgPro20 and IgPro10 in subjects with dcSSc were consistent with that in approved indications such as chronic inflammatory demyelinating polyneuropathy (CIDP), including a relatively low ISR rate for IgPro20. PK profiles and bioavailability were also similar to other indications, as addressed further below.

### B. Overall Study Design and Plan: Description

Subjects with a diagnosis of dcSSc with disease duration ≤5 years and skin thickness scores of ≥15 to ≤45 as measured by mRSS were eligible to enroll in the study. During the screening period, investigators evaluated the eligibility of potential subjects utilizing the 2013 European League Against Rheumatism/ACR criteria (van den Hoogen et al., Ann. Rherm. Dis. 72(11): 1747-55 (2013)) for SSc and diffuse features (LeRoy et al., J. Rheumatology 15(2): 202-5 (1988); LeRoy et al., J. Rheumatology 28(7): 1573-6 (2001)) to accurately confirm their classification as dcSSc. All eligible subjects were randomized (1:1) to Sequence A (IgPro20-IgPro10 treatment sequence) or Sequence B (IgPro10-IgPro20 treatment sequence). Each subject was to complete 2 treatment periods (Treatment Period 1 and Treatment Period 2; 16 weeks each), with up to 40 weeks (including screening) of study duration for an individual subject.

Subjects in Sequence A received a total dose of 0.5 g/kg IgPro20 over 2 sessions per week every week in Treatment Period 1, and a total dose of 2 g/kg IgPro10 over 2 to 5 sessions on consecutive days every 4 weeks in Treatment Period 2. Subjects in Sequence B received a total dose of 2 g/kg IgPro10 over 2 to 5 sessions on consecutive days every 4 weeks in Treatment Period 1 and a total dose of 0.5 g/kg IgPro20 over 2 sessions per week every week in Treatment Period 2. The weekly 0.5 g/kg body weight dose of IgPro20 is equivalent to an every 4 week dose of IgPro10 (2 g/kg body weight) if using a 1:1 conversion. This was the highest possible IgPro20 SC dose because of volume limitation and non-characterized safety in SSc subjects with skin impairment.

### 1. Study Design

The crossover design with 2 sequences was chosen to provide a reliable estimate of IgPro20 relative bioavailability because IgPro20 and IgPro10 treatment differences are measured within a subject rather than between subjects with SSc; both inter- and intraindividual variability were characterized. This study design also enabled investigation of the potential differences in the relative bioavailability of IgPro20 (SCIG) when given before or after IgPro10 (IVIG) in SSc subjects. During both Treatment Period 1 and Treatment Period 2 in each sequence, several PK samples to measure IgG trough concentrations were collected to assess the steady state of IgPro20 or IgPro10 and carry-over effects on Treatment Period 2 from Treatment Period 1. In addition, PK samples were frequently collected over the last dose period to fully characterize the PK of IgPro20 and IgPro10, respectively, for each treatment period in this patient population.

The 16 weeks of treatment for both IgPro20 and IgPro10 allowed for the assessment of safety and preliminary efficacy signals as well as assessment of IgPro20 and IgPro10 PK profiles at the steady state. The duration of PK assessments was based on IgG trough concentrations measured in the pivotal phase 3 study in subjects with CIDP (IgPro20_3003 study), indicating that IgPro20 and IgPro10 would reach steady state within approximately 8 weeks. Importantly, several trough concentrations were determined for IgPro20 and IgPro10 in this study to confirm that steady state was reached before the full PK profile of IgPro20 or IgPro10 was assessed in subjects with SSc.

The study did not have a washout period since the full PK profile of IgPro10 in the respective treatment period of each sequence was obtained at the end of the treatment period, when it was assumed that steady state would have been reached. The relative bioavailability of IgPro20 was evaluated based on the dose-normalized AUC₀₋ₜₐᵤ of IgPro20 and IgPro10 without the need of a washout interval. Moreover, no washout period between the 2 treatments shortened the study duration, making it potentially less burdensome for SSc subjects.

### 2. Pharmacokinetics Assessments

Serum samples for IgG trough level determination were collected at several study visits. Additional blood samples for rich PK sampling of IgG levels were collected at the end of each treatment period to calculate PK parameters (AUC₀₋ₜₐᵤ, AUC₀₋ₗₐₛₜ, Cₘₐₓ, C_{trough}, and time of maximum concentration [Tmax]). IgG concentrations from PK sampling were listed for individual subjects and summarized by nominal (planned) time points. Individual concentration-time profiles and mean (±SD) profiles following the first infusion of the last infusion cycle in each Treatment Period and Treatment Sequence were plotted using actual time points for individual plots and nominal (planned) time points for mean profiles.

Noncompartmental PK analysis was performed by ICON Early Clinical & Bioanalytical using Phoenix WinNonLin (Build 8.0.0.3176). Only concentration-time profiles following the first infusion of the last infusion cycle in each Treatment Period and Treatment Sequence were used to calculate the PK parameters. The PK parameters (AUC₀₋ₜₐᵤ, AUC₀₋ₗₐₛₜ, Cₘₐₓ, C_{trough}, and Tₘₐₓ) were derived using actual sampling collection times and from serum IgG levels using a noncompartmental analysis approach. The serum PK parameters were listed and summarized by Treatment Period in each Treatment Sequence with mean (±SD) and geometric mean (geometric % CV and 95% CI) for all PK parameters except Tmax based on the PK set. C_{trough} was summarized by each time point by treatment in each Treatment Sequence. Tₘₐₓ was listed only.

Relative bioavailability was analyzed based on Baseline-corrected C_{trough} and dose-normalized Baseline-corrected C_{trough}. Population bioavailability was assessed using Mixed Model Repeated Measures on log-transformed dose-normalized Baseline-corrected area under the curve from beginning to end of the dosing period (C_{trough}) following administration of the first does of IgPro20 to the last week of dosing for the IgPro20. Relative bioavailability was calculated as the geometric mean ratio of area under the serum IgG concentration-time curves within a dosing duration (area under the concentration-time curve from time point 0 to tau [AUC₀₋ₜₐᵤ]) following administration of the first dose of IgPro20 or IgPro10 in the last week of dosing for IgPro20. The model included treatment, Treatment Period, and treatment-by-Treatment-Period interaction as fixed effects with an unstructured covariance matrix. Geometric Mean Ratio and corresponding 90% CI derived from the statistical model were used to assess the relative bioavailability of IgPro20 based on dose-normalized Baseline-corrected AUC₀₋ₜₐᵤ. Specifically, relative bioavailability was calculated as: (AUC₀₋ₜₐᵤ IgPro20 (SC)/dose of IgPro20 (SC))/(AUC0-tau of IgPro10 (IV)/dose of IgPro10 (IV)).

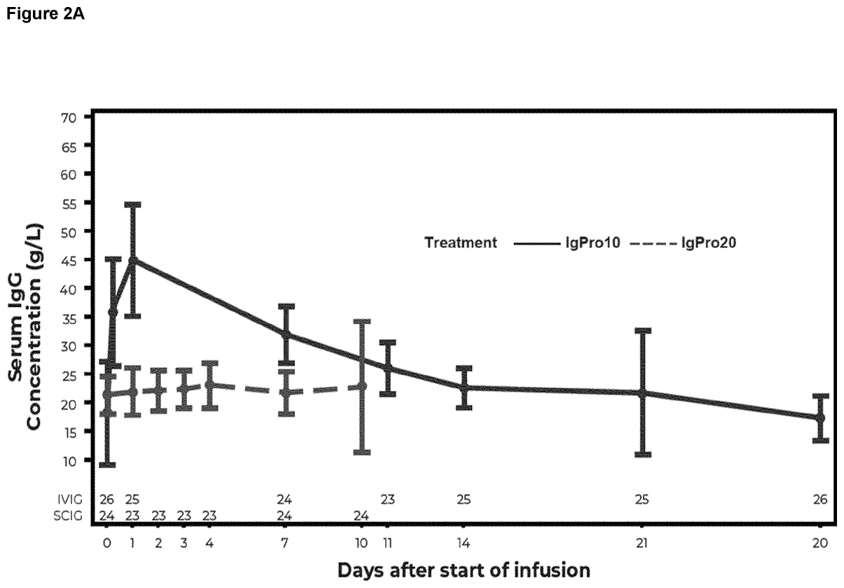

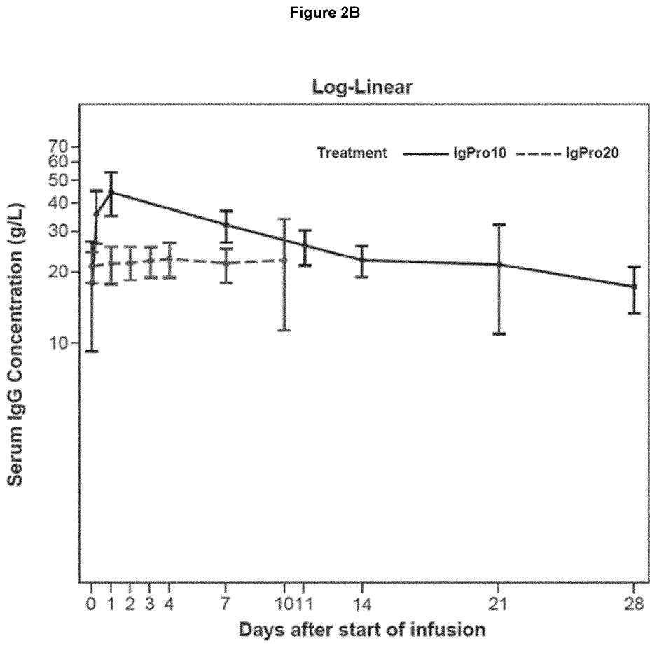

Following a review of the serum IgG concentrations data, 2 outlying observations were identified, 1 for each treatment (IgPro10 outlier, Sample AP21; IgPro20 outlier, Sample BH22). In each case, the IgG concentration in the sample was much higher than the preceding sample, and as a consequence, these sample points had a disproportionate effect on the estimated parameters. Both samples were excluded from the PK parameter calculations as per CSL guidelines. No other data were excluded from the PK analysis. The mean (+/−standard deviation) of serum IgG concentration-time profiles following the first infusion of the last infusion cycle are presented by treatment in FIG. 2A (linear scale) and FIG. 2B (log-linear scale), where the solid line shows data for the IgPro10 treatment and the dashed line for the IgPro20 treatment.

The serum PK parameters of IgPro10 and IgPro20 are summarized by treatment and overall in Table 1 and relative bioavailability is presented in Table 2.

TABLE 1
Serum Pharmacokinetic Parameters of
IgG Following IgPro10 and IgPro20

| | |
|---|---|
| IgPro10 | IgPro20 |

| | | | |
|---|---|---|---|
| | GeoMean | | GeoMean |
| n | (geomSD) | n | (geomSD) |

AUC₀₋ₜₐᵤ (h*g/L)

| | | | | |
|---|---|---|---|---|
| Period 1 | 12 | 17672.03 | 12 | 3835.68 |
| | | (1.124) | | (1.212) |
| Period 2 | 12 | 16942.66 | 11 | 3581.08 |
| | | (1.199) | | (1.116) |
| Overall | 24 | 17303.50 | 23 | 3711.73 |
| | | (1.163) | | (1.172) |

Baseline-corrected AUC₀₋ₜₐᵤ (h*g/L)

| | | | | |
|---|---|---|---|---|
| Period 1 | 12 | 9894.94 | 12 | 1539.39 |
| | | (1.288) | | (1.328) |
| Period 2 | 12 | 8021.36 | 12 | 1648.69 |
| | | (1.324) | | (1.410) |
| Overall | 24 | 8909.03 | 24 | 1593.10 |
| | | (1.326) | | (1.364) |

AUC₀₋ₗₐₛₜ (h*g/L)

| | | | | |
|---|---|---|---|---|
| Period 1 | 12 | 17349.72 | 12 | 5361.61 |
| | | (1.145) | | (1.218) |
| Period 2 | 13 | 16520.48 | 12 | 4898.02 |
| | | (1.207) | | (1.168) |
| Overall | 25 | 16913.45 | 24 | 5124.58 |
| | | (1.178) | | (1.197) |

Cₘₐₓ (g/L)

| | | | | |
|---|---|---|---|---|
| Period 1 | 12 | 44.996 | 12 | 24.237 |
| | | (1.2418) | | (1.1984) |
| Period 2 | 13 | 47.142 | 12 | 23.203 |
| | | (1.2471) | | (1.1089) |
| Overall | 25 | 46.099 | 24 | 23.714 |
| | | (1.2405) | | (1.1570) |

AUC₀₋ₜₐᵤ = area under the concentration from time point 0 to tau;
AUC₀₋ₗₐₛₜ = area under the concentration from time point 0 to the last quantifiable time point;
Cₘₐₓ = maximum concentration;
GeoMean = geometric mean;
geomSD = geometric standard deviation;
IgG = Immunoglobulin G;
n = number of subjects

TABLE 2
Population Relative Bioavailability

| | | | | Relative |
|---|---|---|---|---|
| | | | | Bioavailability |
| IgPro10 | | IgPro20 | | Geometric |
| | GeoMean | | GeoMean | Mean Ratio |
| n | (geomSD) | n | (geomSD) | (90% CI) |

Dose-Normalized Baseline-corrected AUC₀₋ₜₐᵤ (h*g/L)

| | | | | | |
|---|---|---|---|---|---|
| Sequence A | 12 | 52.019 | 12 | 42.007 | 0.831 |
| | | (1.4399) | | (1.4636) | (0.7343, 0.9396) |
| Sequence B | 12 | 69.657 | 12 | 47.810 | 0.698 |
| | | (1.2723) | | (1.3289) | (0.6235, 0.7804) |
| Overall | 24 | 60.195 | 24 | 44.814 | 0.761 |
| | | (1.4007) | | (1.3984) | (0.7033, 0.8232) |

AUC₀₋ₜₐᵤ = area under the curve from time point 0 to tau;
CI = confidence interval;
GeoMean = geometric mean;
geomSD = geometric standard deviation;
n = number of subjects

At Week 1 (Baseline), mean (SD) serum IgG concentrations were 13.271 g/L (4.3011) and 12.349 g/L (4.2912) in Sequence A (IgPro20/IgPro10) and Sequence B (IgPro10/IgPro20), respectively. Mean trough serum IgG concentrations in the IgPro20 Periods in both Sequence A and B were higher than the mean trough serum IgG concentrations in the IgPro10 Periods, ranging from 22.175 to 23.821 g/L (for IgPro20 in Sequence A, when IgPro20 is administered first) and from 20.590 to 22.042 g/L (for IgPro20 in Sequence B, when IgPro20 is administered second), compared to from 17.322 to 17.945 g/L (for IgPro10 in Sequence B, when IgPro10 is administered first) and from 17.080 to 19.559 g/L (for IgPro10 in Sequence A, when IgPro10 is administered second). Mean Cₘₐₓ was higher following IgPro10 administration compared to IgPro20 administration. Population relative bioavailability, based on dose-normalized, Baseline-corrected AUC₀₋ₜₐᵤ, was 0.761 (or 76.1%). The relative bioavailability of IgPro20 was higher in Sequence A (IgPro20/IgPro10) than in Sequence B (IgPro10/IgPro20), and the geometric mean ratios (90% CI) were 0.831 and 0.698, respectively.

The geometric mean (geometric SD) AUC0-tau and Cmax were 3711.73 h*g/L (1.172) and 23.714 g/L (1.1570) for IgPro20 and 17,303.50 h*g/L (1.163) and 46.099 g/L (1.2405) for IgPro10, respectively. Relative bioavailability of IgPro20, based on dose-normalized, Baseline-corrected AUC₀₋ₜₐᵤ was 0.761 (90% CI: 0.7033, 0.8232).

### 3. Efficacy Assessments

Various measures of efficacy were employed for the study, including the American College of Rheumatology Composite Response Index in Diffuse Cutaneous Systemic Sclerosis (ACR CRISS) step 1 and step 2, modified Rodnan Skin Score (mRSS), Scleroderma Health Assessment Questionnaire (SHAQ) Visual Analog Scale (VAS) and Health Assessment Questionnaire Digital Impairment (HAQ DI), UCLA Scleroderma Consortium Gastrointestinal Tract(UCLA SCTC GIT) version 2.0, Physician's Global Assessment (MDGA), and Patient Global Assessment (PGA) tests, as well as assessments of muscle strength, tender or swollen joints and level of friction in joints.

The ACR-CRISS incorporates multisystem involvement in dcSSc and is calculated as a 2-step process. The first step evaluates clinically significant decline in renal or cardiopulmonary involvement that is related to SSc and that requires treatment. If a patient develops any of the 4 following outcomes after treatment, they are considered to be not improved in step 1 (i.e., to meet the step 1 criteria): a) a new scleroderma renal crisis; b) a decline in FVC of 15% or more of predicted (relative), confirmed by another FVC % within one month, high-resolution computed tomography to confirm interstitial lung disease and FVC % below 80% of predicted attributable to SSc, c) a new decline of left ventricular ejection fraction to 45% or less requiring treatment attributable to SSc, or d) new onset of pulmonary arterial hypertension (defined as mean pulmonary artery pressure ≥25 mmHg at rest, an end-expiratory pulmonary wedge pressure s 15 mmHg and a pulmonary vascular resistance >3 Wood units), which is attributable to SSc that requires treatment.

No subjects met the ACR-CRISS Step 1 criteria. A Serious Adverse Event (SAE) of chest pain was reported for 1 subject, and this was recorded in the database as meeting the ACR-CRISS Step 1 criteria “new onset of left ventricular failure (left ventricular ejection fraction ≤45%) requiring treatment.” The event was recorded as meeting ACR-CRISS Step 1 in the database. The subject was ultimately diagnosed with possible microvascular coronary disease based on clinical picture and exclusion of coronary artery disease. The contribution of SSc-related microangiopathy could not be excluded or confirmed; however, the subject had other risk factors including dyslipidemia and obesity.

The second step of the ACR CRISS was conducted based on changes in the mRSS, FVC % predicted, PGA, MDGA, and HAQ-DI. The mean (SD) Step 2 components at Baseline and the mean changes from Baseline at week 17 before infusion and at week 32/End of Treatment are summarized in Table 3.

In total, the number of subjects who were responders based on ACR-CRISS (defined as meeting ACR-CRISS>0.6) over week 1 to week 17 was 4 (14.8%), including 3 subjects in Sequence A (IgPro20/IgPro10) and 1 subject in Sequence B (IgPro10/IgPro20); over Weeks 1 to 32, 12 (44.4%) subjects were responders (met ACR-CRISS>0.6).

Improvements in mean mRSS Total Score were observed following each treatment at week 17 and week 32. Mean Forced Vital Capacity (FVC, in %) predicted values at week 17 and week 32 were similar to those observed at Baseline. Improvements from Baseline in the mean results of the MDGA were observed at week 17 and week 32 in IgPro20 Periods. Mean results of the PGA at week 17 and week 32 were similar to those observed at Baseline. Mean HAQ-DI values at week 32 were similar to those observed at Baseline. Out of 27 subjects, 11 (40.7%) subjects were responders based on mRSS in weeks 1 to 17, and 18 (66.7%) were responders over weeks 1 to 32, applying an mRSS response definition of change from Reference (Baseline) Visit ≤−5 and percentage change from Reference (Baseline) Visit ≤−25%. Out of 27 subjects, 13 (48.1%) subjects were responders in weeks 1 to 17, and 20 (74.1%) were responders over weeks 1 to 32, applying an mRSS response definition of change from Reference (Baseline) Visit ≤−4 and percentage change from Reference (Baseline) Visit ≤−20%. Overall, the mRSS Total Score at baseline, before treatment, ranged from 20 to 25 and decreased by a value of between 3 and 8 following the 17 weeks of IgPro20 treatment.

TABLE 3
Mean (SD) Changes from Baseline in ACR-CRISS Components
Step 2 by Sequence and Total (ITT Analysis Set)

| Sequence A | | Sequence B | | | |
|---|---|---|---|---|---|
| (IgPro20/IgPro10) | | (IgPro10/IgPro20) | | Total | |
| (N = 13) | | (N = 14) | | (N = 27) | |
| n | Mean (SD) | n | Mean (SD) | n | Mean (SD) |

mRSS Total Score (range: 0 to 51, higher is worse, negative change is improvement)

| | | | | | | |
|---|---|---|---|---|---|---|
| Baseline | 13 | 23.8 | 14 | 25.0 | 27 | 24.4 |
| | | (5.83) | | (7.08) | | (6.42) |

Change from Baseline

| | | | | | | |
|---|---|---|---|---|---|---|
| Week 17, before infusion | 10 | −5.7 | 9 | −3.4 | 19 | −4.6 |
| | | (4.76) | | (4.28) | | (4.56) |
| Week 32, End of | 13 | −7.6 | 14 | −7.5 | 27 | −7.6 |
| Treatment | | (6.33) | | (5.39) | | (5.75) |

FVC % Predicted (higher is better, positive change is improvement)

| | | | | | | |
|---|---|---|---|---|---|---|
| Baseline | 13 | 92.29 | 14 | 94.03 | 27 | 93.19 |
| | | (15.480) | | (15.542) | | (15.237) |

Change from Baseline

| | | | | | | |
|---|---|---|---|---|---|---|
| Week 17, before infusion | 10 | −0.28 | 13 | −0.03 | 23 | −0.14 |
| | | (3.138) | | (4.929) | | (4.159) |
| Week 32, End of | 13 | −1.58 | 14 | −0.63 | 27 | −1.09 |
| Treatment | | (5.011) | | (5.447) | | (5.163) |

Patient's Global Assessment (range: 0 to 10, higher is worse, negative change is
improvement)

| | | | | | | |
|---|---|---|---|---|---|---|
| Baseline | 12 | 3.5 | 14 | 4.3 | 26 | 3.9 |
| | | (1.93) | | (1.54) | | (1.74) |

Change from Baseline

| | | | | | | |
|---|---|---|---|---|---|---|
| Week 17, before infusion | 10 | 0.0 | 12 | −0.7 | 22 | −0.4 |
| | | (1.89) | | (2.23) | | (2.06) |
| Week 32, End of | 12 | −0.6 | 14 | −0.7 | 26 | −0.7 |
| Treatment | | (1.62) | | (2.09) | | (1.85) |

Physician Global Assessment (range: 0 to 10, higher is worse, negative change is
improvement)

| | | | | | | |
|---|---|---|---|---|---|---|
| Baseline | 11 | 3.4 | 11 | 3.8 | 22 | 3.6 |
| | | (2.11) | | (1.83) | | (1.94) |

Change from Baseline

| | | | | | | |
|---|---|---|---|---|---|---|
| Week 17, before infusion | 7 | −1.0 | 6 | −0.3 | 13 | −0.7 |
| | | (2.77) | | (1.51) | | (2.21) |
| Week 32, End of | 10 | −0.3 | 11 | −1.3 | 21 | −0.8 |
| Treatment | | (0.82) | | (2.05) | | (1.63) |

HAQ-DI (range: 0 to 3, higher is worse, negative change is improvement)

| | | | | | | |
|---|---|---|---|---|---|---|
| Baseline | 12 | 0.5833 | 14 | 0.9196 | 26 | 0.7644 |
| | | (0.73727) | | (0.77461) | | (0.76185) |

Change from Baseline

| | | | | | | |
|---|---|---|---|---|---|---|
| Week 17, before infusion | 10 | 0.0000 | 12 | −0.2083 | 22 | −0.1136 |
| | | (0.48233) | | (0.33850) | | (0.41352) |
| Week 32, End of | 12 | −0.0417 | 14 | −0.0625 | 26 | −0.0529 |
| Treatment | | (0.40005) | | (0.33880) | | (0.36086) |

ACR-CRISS = American College of Rheumatology Composite Response Index in diffuse cutaneous Systemic Sclerosis;
FVC = Forced vital capacity;
HAQ-DI = Health Assessment Questionnaire-Disability Index;
ITT = Intent to Treat;
mRSS = Modified Rodnan Skin Score;
N = number of subjects;
n = number of observations;
SD = Standard deviation

TABLE 4
Mean (SD) Exploratory Efficacy Assessments

| Sequence A | | Sequence B | | | |
|---|---|---|---|---|---|
| (IgPro20/IgPro10) | | (IgPro10/IgPro20) | | Total | |
| (N = 13) | | (N = 14) | | (N = 27) | |
| n | Mean (SD) | n | Mean (SD) | n | Mean (SD) |

SHAQ VAS (Overall disease) (range 0 to 3, higher is worse)

| | | | | | | |
|---|---|---|---|---|---|---|
| Baseline | 12 | 1.243 | 14 | 1.234 | 26 | 1.238 |
| | | (0.8870) | | (0.6168) | | (0.7376) |
| Week 17, before infusion | 11 | 1.110 | 12 | 1.213 | 23 | 1.163 |
| | | (0.9542) | | (0.6974) | | (0.8123) |
| Week 32, End of | 13 | 0.928 | 14 | 1.114 | 27 | 1.024 |
| Treatment | | (0.9930) | | (0.5688) | | (0.7911) |

UCLA SCTC GIT 2.0 total score (range 0 to 3, higher is worse)

| | | | | | | |
|---|---|---|---|---|---|---|
| Baseline | 12 | 0.375 | 14 | 0.408 | 26 | 0.393 |
| | | (0.4385) | | (0.4886) | | (0.4572) |
| Week 17, before infusion | 11 | 0.336 | 12 | 0.400 | 23 | 0.370 |
| | | (0.3589) | | (0.4965) | | (0.4277) |
| Week 32, End of | 13 | 0.363 | 14 | 0.441 | 27 | 0.403 |
| Treatment | | (0.3210) | | (0.5722) | | (0.4614) |

SSPRO total score (range 0 to 100, higher is better)

| | | | | | | |
|---|---|---|---|---|---|---|
| Baseline | 12 | 30.710 | 14 | 44.643 | 26 | 38.212 |
| | | (27.5265) | | (25.2668) | | (26.7496) |
| Week 17, before infusion | 11 | 24.747 | 12 | 38.040 | 23 | 31.683 |
| | | (24.7279) | | (21.3035) | | (23.4724) |
| Week 32, End of | 13 | 22.009 | 12 | 35.847 | 27 | 29.184 |
| Treatment | | (27.2170) | | (19.6668) | | (24.1853) |

Number of swollen joints (range 0 to 28, higher is worse)

| | | | | | | |
|---|---|---|---|---|---|---|
| Baseline | 13 | 0.5 | 14 | 0.0 | 27 | 0.2 |
| | | (1.20) | | (0.00) | | (0.85) |
| Week 17, before infusion | 12 | 0.1 | 13 | 0.0 | 25 | 0.0 |
| | | (0.29) | | (0.00) | | (0.20) |
| Week 32, End of | 13 | 0.2 | 14 | 0.3 | 27 | 0.3 |
| Treatment | | (0.83) | | (1.07) | | (0.94) |

Number of tender joints (range 0 to 28, higher is worse)

| | | | | | | |
|---|---|---|---|---|---|---|
| Baseline | 13 | 1.5 | 14 | 2.6 | 27 | 2.1 |
| | | (2.70) | | (3.69) | | (3.23) |
| Week 17, before infusion | 12 | 2.2 | 13 | 1.5 | 25 | 1.8 |
| | | (2.69) | | (2.33) | | (2.48) |
| Week 32, End of | 13 | 0.3 | 14 | 0.9 | 27 | 0.6 |
| Treatment | | (0.85) | | (2.46) | | (1.86) |

Manual muscle test (total score)

| | | | | | | |
|---|---|---|---|---|---|---|
| Baseline | 13 | 149.3 | 14 | 134.3 | 27 | 141.5 |
| | | (1.11) | | (32.44) | | (24.19) |
| Week 17, before infusion | 11 | 143.0 | 13 | 136.5 | 24 | 139.5 |
| | | (16.09) | | (26.09) | | (21.88) |
| Week 32, End of | 13 | 139.3 | 14 | 135.8 | 27 | 137.5 |
| Treatment | | (22.19) | | (25.24) | | (23.43) |

N = number of subjects;
n = number of observations;
UCLA SCTC GIT 2.0 = University of California, Los Angeles, Scleroderma Clinical Trial Consortium Gastrointestinal Tract 2.0;
SD = Standard deviation;
SHAQ = Scleroderma Health Assessment Questionnaire;
SSPRO = Scleroderma skin patient-reported outcome;
VAS = Visual Analog Scale

At weeks 17 and 32, a decrease (improvement) in mean SHAQ overall disease scores was observed (Table 4). Small improvements from Baseline were generally observed for each of the SHAQ VAS items (pain, intestinal disease, breathing problems, Raynaud's syndrome, finger ulcers, and overall disease). The mean total scores and the mean scores for each item (reflux, distension, soilage, diarrhea, social functioning, emotional wellbeing, and constipation) were generally similar before the start of and at the end of the assessment period.

The MDGA score decreased by about 0.3 to 1.3 from an initial score of 3.4 to 3.8 in the two groups of patients, or decreased by an average of 0.8 from an initial average score of 0.8. The PGA score decreased from 0 to 0.7 after 17 weeks of treatment, with an average initial score of 3.9 decreasing by an average of 0.4 to 0.7 over the treatment time period. The UCLA SCTC GIT 2.0 score did not increase on average in the trial subjects. (See Tables 3-4.)

The mean total Scleroderma Skin Patient-reported Outcome (SSPRO) score at week 17, before infusion, and at week 32/End of Treatment decreased from Baseline (indicating a worsening of symptoms) following both Treatment Sequences (Table 4). Decreases from Baseline (indicating a worsening of symptoms) were observed in each of the SSPRO components: physical function, social effects, emotional effects, and physical function. The SSPRO assesses skin-specific health-related quality of life in SSc patients. (A. Man et al., Ann. Rheum. Dis. 76: 1374-80 (2017).) The test has 18 items that represent 4 health-related quality of life scales: physical symptoms, social effects, emotional effects, and physical function. Each is scored on a 7-point Likert scale and the total score is normalized to a 0-100 scale.

Investigators also evaluated 28 joints for swelling: 10 proximal interphalangeal joints of the fingers, 10 metacarpophalangeal joints, wrists, elbows, shoulders, and knees. The number of subjects with ≥4 swollen joints was low, observed in 1 (3.7%) subject at Baseline and 1 (3.7%) subject at Week 32/End of Treatment (Table 14.2.5.5). The number of swollen joints was low, with a total mean (SD) of 0.2 (0.85) at Baseline. The mean (SD) total number of swollen joints was similar throughout the study; 0.0 (0.20) at Week 17, before infusion, and 0.3 (0.94) at Week 32/End of Treatment.

Investigators also evaluated 28 joints for tenderness: 10 proximal interphalangeal joints of the fingers, 10 metacarpophalangeal joints, wrists, elbows, shoulders, and knees. A total of 6 (22.2%) subjects had 4 tender joints at Baseline. At Week 17, the number of subjects with 4 tender joints was 6 (22.2%), and at Week 32/End of Treatment, the number of subjects with 4 tender joints had decreased to 1 (3.7%). The number of tender joints was low, with a total mean (SD) of 2.1 (3.23) at Baseline. The mean number of tender joints decreased during the study; 1.8 (2.48) at Week 17, before infusion, and 0.6 (1.86) at Week 32/End of Treatment, a change from Baseline of −0.4 (3.03) and −1.4 (2.36), respectively.

Tendon friction rubs through simple bilateral palpation at the extensor and flexor tendons of the fingers and wrists, and tendons over the elbow, knees, ankles, shoulders, and scapulas were also assessed. At Baseline, 1 (3.7%) subject had tendon friction rubs; this increased to 4 (14.8%) at Week 17, before infusion, and was 3 (11.1%) at Week 32/End of Treatment.

MMT total scores were also obtained. The total mean (SD) of the manual muscle test (MMT) total scores was 141.5 (24.19). This decreased (indicating a worsening of symptoms) slightly during the study and was 139.5 (21.88) at Week 17, before infusion, a −1.0 (13.63) change from Baseline, and 137.5 (23.43) at Week 32/End of Treatment, a −4.0 (18.48) change from Baseline. When summarized by sequence, a decrease (worsening) in the mean was observed for Sequence A, and a slight increase (improvement) was observed for Sequence B.

For the ACR-CRISS Components (mRSS, FVC % predicted, PGA, MDGA, and HAQ-DI), trends for improvement with IgPro20 were observed on mRSS and MDGA (clinician reported measures). Improvements were observed over the first treatment periods and sustained following crossover to IgPro10 with mRSS and in each treatment period on IgPro20 regardless of sequence in MDGA. Trends for improvement with IgPro20 and IgPro10 were also seen in SHAQ VAS for overall disease, regardless of sequence. For example, SHAQ VAS overall disease score on average decreased from 1.2 at baseline to 1.0 following treatment.

The UCLA SCTC GIT 2.0 survey was also conducted. The mean total scores and the mean scores for each item in the UCLA SCTC GIT were generally similar before the start of and at the end of the assessment period.

1. A method of treating systemic sclerosis (SSc) in a human subject, comprising subcutaneous administration of a liquid human polyclonal immunoglobulin preparation comprising 16-22% w/v immunoglobulin, wherein the preparation is administered once, twice, or three times per week in order to achieve a weekly dose of 0.1 g/kg to 1 g/kg.

2. The method of claim 1, wherein the preparation is administered twice per week.

3. The method of claim 1, wherein the preparation comprises 20-22% immunoglobulin.

4. The method of claim 1, wherein the immunoglobulin is derived from pooled plasma of more than 1000 human donors.

5. The method of claim 1, wherein the preparation further comprises a stabilizer comprising an amino acid.

6. The method of claim 5, wherein the amino acid is selected from proline, glycine, or arginine.

7. The method of claim 5, wherein the stabilizer further comprises a surfactant.

8. The method of claim 1, wherein the preparation:
(a) has a pH of 4.6 to 5.2;
(b) comprises no carbohydrate stabilizers;
(c) comprises no more than 50 g/mL IgA; and/or
(d) comprises no preservatives.

9. (canceled)

10. The method of claim 1, wherein the SSc is diffuse cutaneous systemic sclerosis (dcSSc) or limited cutaneous SSc (lcSSc).

11. The method of claim 1, wherein the SSc is dcSSc.

12. The method of claim 10, wherein the subject does not have dcSSc at the subcutaneous infusion site.

13. The method of claim 1, wherein the method further comprises administration of at least one additional therapeutic agent.

14. The method of claim 13, wherein the at least one additional therapeutic agent:
(a) does not comprise an antibody therapeutic agent; and/or
(b) does not comprise an anti-CD20 antibody, an anti-IL6 antibody, and/or an anti-IL6 receptor antibody.

15. (canceled)

16. The method of claim 1, wherein:
(a) the subject has not previously received treatment with an anti-CD20 antibody, an anti-IL6 antibody, and/or an anti-IL6 receptor antibody;
(b) the subject has not been diagnosed with a rheumatic autoimmune disease other than SSc;
(c) the subject, prior to the start of treatment, does not have a history of dermatitis, eczema or psoriasis;
(d) the subject has a modified Rodnan skin score of 15-45 prior to treatment with the preparation;
(e) wherein the subject experiences no more than a mild or moderate infusion site skin reaction following administration with the preparation; and/or
(f) the subject does not experience an infusion site reaction such as infusion site pain or infusion site swelling within the first month of treatment, or within the first three months of treatment.

17. (canceled)

18. The method of claim 2, wherein:
(a) the preparation is administered twice per week in order to achieve a weekly dose of 0.5 g/kg;
(b) the preparation is administered for a period of at least one month; and/or
(c) the preparation is subcutaneously administered using an infusion pump device.

19.-24. (canceled)

25. The method of claim 1, wherein a volume of 10 mL to 100 mL is subcutaneously administered in each dose.

26. The method of claim 1, wherein the preparation is administered for a period of at least one month, and wherein the subject, after one month, after 3 months, or after 4 months of treatment:
a. has a trough serum IgG concentration of at least 18 g/L;
b. has an mRSS value that is reduced by at least 3, or that is reduced by at least 15% compared to baseline;
c. has a SHAQ VAS that is decreased by at least 5% compared to baseline;
d. has a PGA that is decreased by at least 5% compared to baseline;
e. has an MDGA that is decreased by at least 5% compared to baseline;
f. has reduced pain, swelling and/or friction in at least one joint compared to baseline; and/or
g. does not have an increase in UCLA SCTC GI 2.0 score compared to baseline.

27. A liquid human polyclonal immunoglobulin preparation comprising 16-22% w/v immunoglobulin, wherein the preparation is formulated for subcutaneous administration, and administration once, twice, or three times per week to a human subject with systemic sclerosis (SSc) achieves a weekly dose of 0.1 g/kg to 1 g/kg.

28. (canceled)

29. The method of claim 6, wherein the amino acid is proline.

30. The method of claim 29, wherein the proline is at a concentration of 100-500 mM.
